(19) United States
(12) Patent Application Publication
(10) Pub. No.: US 2022/0193272 A1
(43) Pub. Date: Jun. 23, 2022

(54) Bleeding Detection Method

(71) Applicant: Ethicon, Inc., Somerville, NJ (US)

(72) Inventors: Ashley DeAnglis, Skillman, NJ (US); Salim A. Ghodbane, Piscataway, NJ (US); Sridevi Dhanaraj, Raritan, NJ (US); Brian Aitken, Somerville, NJ (US)

(21) Appl. No.: 17/546,378

(22) Filed: Dec. 9, 2021

Related U.S. Application Data
(60) Provisional application No. 63/128,594, filed on Dec. 21, 2020.

(51) Int. Cl. A61K 49/00

(52) U.S. Cl. CPC A61K 49/0056; A61K 49/0013; A61K 49/0069

(57) ABSTRACT

Disclosed are devices for detection of bleeding during surgical procedures and uses thereof. Also disclosed are methods of localizing a bleeding site during surgical procedures, and methods for evaluating an intensity of bleeding during the surgical procedures. The detection of bleeding is based, inter alia, on the presence of thrombin activity in a bleeding site.

### FIELD OF THE INVENTION

The present invention relates, inter alia, to a method and device for localizing bleeding and/or determining bleeding intensity, e.g. in a surgical field, and uses thereof.

### BACKGROUND OF THE INVENTION

Tissue trauma and damage to a blood vessel results in bleeding. The physiological response to bleeding involves vascular endothelial cells, platelets and coagulation proteins. After transient vasoconstriction after blood vessel injury, platelets begin to accumulate at the site of vessel disruption. Platelet binding is followed by platelet activation which further recruits additional platelets to the site of vessel injury thus forming a platelet plug. Activated platelets assist in the generation of active coagulation enzymes by providing an ideal surface for the localization of clotting factors. This process is commonly called the “coagulation cascade” and leads to conversion of prothrombin, an inactive zymogen, to thrombin, an active enzyme that is responsible for conversion of soluble fibrinogen to an insoluble fibrin clot.

Under physiological conditions thrombin will be generated after tissue trauma and vessel injury. The amount of thrombin generated will depend on many factors, but it is primarily driven by the amount of tissue factor exposed at the site of vessel injury. After generation of thrombin and formation of a fibrin clot, the coagulation response will be downregulated by the Protein C system and thrombin activity will be reduced by endogenous anticoagulants, e.g., antithrombin III. Additionally, thrombin will be inactivated by absorption onto the fibrin polymer limiting its activity in the solution phase.

In patients with diseased tissue, obese patients, or patients with distorted anatomy (e.g., revision surgery) visualization in a surgical field can be challenging. Regardless of specialty or surgical procedure, it is a fundamental requirement to be able to visualize and protect structures within the surgical field. However, there are some procedures in which visualization is especially problematic including prostatectomies, high colectomies (splenic flexure), radical hysterectomies, nephrectomies, and spinal and cranial operations close to nerve tracks.

Hemorrhages and blood loss significantly aggravate a patient's medical condition. Fast detection of bleeding is critical, and appropriate control of the bleeding is mandatory.

Furthermore, it can be challenging for a surgeon to identify the presence and location of bleeding, especially during laparoscopic/endoscopic procedures. While viewing a procedure on a video screen during a Minimally Invasive Surgery (MIS) procedure, the ability for the surgeon to differentiate whether blood seen in the surgical field is e.g., oozing, or already clotted is reduced.

Currently, to identify potential bleeding, surgeons must carefully visually inspect the surgical field to identify and locate bleeding sites. This careful inspection extends procedural time in the operating room and during MIS procedures is complicated by the limited field of vision and the need to keep scope clean by removing it in and out of the field of inspection. If a surgeon mis-identifies a bleeding site, he/she may apply un-necessary hemostatic agents, or conversely, not treat a bleeding site resulting in blood loss and the associated negative consequences.

Therefore, there is a need for an in vivo method for detecting the presence of bleeding to address at least one of the above problems.

The following are background publications disclosing chromogenic or fluorogenic substrates for determining thrombin activity in an in vitro sample (e.g. blood sample):

U.S. Pat. No. 8,916,356 B2; Chromogenic Substrates in Coagulation and Fibrinolytic Assays, Andrei Z. Budzynski. Laboratory Medicine, July 2001, Number 7, Volume 32; and Product Brochure for S-2238 chromogenic substrate for thrombin, Chromogenix, Instrumentation Laboratory.

### SUMMARY OF THE INVENTION

Oftentimes, visualization of bleeding in a surgical field, e.g. in Minimally Invasive Surgery (MIS) is challenging. Frequently, surgeons may spend prolonged time trying to visualize bleeding thus increasing procedural times. In many cases, consequences of inadequate visualization include mechanical trauma, greater bleeding due to mis-identification of structures, and dissecting into these misidentified anatomical structures.

In one aspect, the invention relates to a method for localizing a bleeding site in a surgical field e.g. in MIS.

In one aspect of the invention, determining thrombin activity is used in a method for localizing a bleeding site in a surgical procedure.

Disclosed is a method of localizing a bleeding site during a surgical procedure in a subject comprising: i) introducing a chromogenic or fluorogenic substrate of thrombin into or onto a potential bleeding site in the body of the subject, and ii) detecting a color or fluorescence signal, thereby localizing the bleeding site in said subject.

In another aspect, the invention relates to a method for determining intensity and or severity of bleeding in a subject during a surgical procedure e.g. in MIS.

Intensity and or severity of bleeding in a subject during a surgical procedure may be determined by assessing the level of thrombin activity.

Disclosed is a method which enables determining the intensity of bleeding during a surgical procedure in a subject comprising introducing a chromogenic or fluorogenic substrate of thrombin into or onto a potential bleeding site in the body of the subject, and determining the presence and intensity of the color or fluorescence signal, thereby determining the presence and intensity of bleeding.

Introducing a chromogenic or fluorogenic substrate of thrombin into or onto a potential bleeding site in the body of the subject may be carried out by applying the substrate on a surface of a potential bleeding site, for example by techniques including but non-limited to spraying, dripping. Other techniques of introducing a chromogenic or fluorogenic substrate of thrombin into or onto a potential bleeding site in the body of the subject may be carried out by intravenous injection (abbreviated as IV administration) of the substrate or by other systemic routes.

Topical administration relates to application to a localized area of the body or to the surface of a body part.

IV administration is a medical technique to deliver fluids directly into a patient's vein.

Intravenous (IV) access is used to administer fluid which must be rapidly distributed throughout the body. A chromogenic or fluorogenic substrate may be mixed into fluids such as normal saline, or dextrose solutions. IV route is a fast way to deliver fluids throughout the body. For this reason, the IV route is commonly preferred in emergency situations or when a fast onset of action is desirable. A loading or bolus dose of a chromogenic or fluorogenic substrate may be given to more quickly increase the concentration of medication in the blood. A bolus dose (or “IV push”) of a chromogenic or fluorogenic substrate may be applied by a syringe containing the a chromogenic or fluorogenic substrate which is connected to an access port in the primary tubing and the a chromogenic or fluorogenic substrate is administered through the port. A bolus may be administered rapidly (with a fast depression of the syringe plunger) or may be administered slowly, over the course of a few minutes. In some cases, a bolus of plain IV fluid (i.e. without a chromogenic or fluorogenic substrate added) is administered immediately after the bolus to further force the a chromogenic or fluorogenic substrate into the bloodstream. This procedure is termed an “IV flush”.

An infusion of a chromogenic or fluorogenic substrate may be used when it is desirable to have a constant blood concentration of a substrate over time.

During surgery intravenous administration of a chromogenic or fluorogenic substrate may be used as a safety measure to ensure that bleeding has stopped.

A way to identify active bleeding under a tissue (e.g. skin tissue, optionally visualization may be carried out visualization through the skin) is e.g. by intravenous administration of a chromogenic or fluorogenic substrate.

Intravenous administration of a chromogenic or fluorogenic substrate may enable the possibility to detect bleeding under a tissue when bleeding may be occurring e.g. in a hematoma (positioned underneath the clot) that could “open” post-operatively. When applied intravenously, the fluorogenic or chromogenic substrate may leak out of the blood vessel with the rest of the blood and react with thrombin generated at the bleeding site.

Typically, but not exclusively the fluorogenic substance may be used for IV administration in view of its sensitivity and lower interference.

Alternatively, a fluorogenic or chromogenic substrate may be introduced into the blood vessels by other means, e.g. by central arterial line.

Typically, a hematoma is a localized bleeding outside of blood vessels, e.g. due to trauma including injury or surgery and may involve blood continuing to seep from broken capillaries.

Said chromogenic or fluorogenic substrate may be (i) immobilized on a porous matrix or a membrane; or (ii) sprayed directly on the potential bleeding site.

A matrix may be porous and may absorb fluid. A matrix may absorb fluid, potentially containing thrombin from the surgical field.

A membrane may be a matrix capable of separating fluid from cells e.g. plasma from cells, plasma from blood cells, and/or plasma from blood cells/whole blood. Such a membrane may be used when separation of plasma from blood cells is needed. The membrane allows the passage of liquid plasma, but filters cells (e.g. large cells). An exemplary membrane is a semipermeable membrane, e.g. for use during hemodialysis.

Advantageously, the method disclosed herein, allows the surgeons to determine the severity of bleeding and e.g. according to the severity to select the appropriate hemostat for a type of bleeding (e.g. oozing/mild bleeding, or severe/challenging bleeding).

A matrix or membrane comprising adsorbed, coated or impregnated chromogenic or fluorogenic thrombin substrate may be used to cover part or all of the area where the surgeon is actively carrying out the surgical procedure tissue or adjacent thereto. A change in color or fluorescent signal appearing in a site of the matrix is indicative of a bleeding site.

Disclosed is also a method of localizing a bleeding site during a surgical procedure in a subject comprising:

i) contacting an absorbent matrix with a potential bleeding site,

ii) removing the matrix from the potential bleeding site,

iii) placing the removed matrix into or onto a detection solution comprising a chromogenic or fluorogenic substrate of thrombin, detecting the appearance of color or fluorescence in the solution, thereby localizing the bleeding site.

In another aspect, disclosed is a method for determining intensity of bleeding during a surgical procedure in a subject, comprising:

i) contacting an absorbent matrix with a potential bleeding site or with a bleeding site,

ii) removing the matrix from the potential bleeding site,

iii) placing the removed matrix into or onto a detection solution comprising a chromogenic or fluorogenic substrate of thrombin and determining the intensity of the color or fluorescence signal, thereby determining intensity of bleeding.

Also, disclosed is a device for localizing a bleeding site and/or determining the intensity of bleeding in a subject during a surgical procedure, the device comprising: an absorbent matrix comprising a chromogenic or fluorogenic substrate of thrombin wherein typically, the matrix is impermeable to red blood cells. The device may be used during a surgical procedure and at a potential bleeding site.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawing in detail, it is stressed that the particulars shown are by way of example and for purposes of illustrative discussion of embodiments of the invention. In this regard, the description taken with the drawing makes apparent to those skilled in the art how embodiments of the invention may be practiced.

In the drawing:

FIG. 1 presents a photographic image showing thrombin activity in a cellulose matrix coated with a chromogenic substrate (left) or in the absence a chromogenic substrate (right), after spraying the matrix with thrombin on both sides.

FIG. 2 presents a photographic image showing thrombin activity on chromogenic substrate using a Dip Stick dabbed onto a tissue surface that is suspected of bleeding (left) followed by introduction on a solution comprising a chromogenic substrate. The amount of the thrombin present in the dip stick can be quantified by comparison with a standard curve obtained by running similar tests with known thrombin concentrations (right).

FIG. 3A-3C

3A presents a photographic image showing detection of chromogenic substrate product in plasma activated by a thromboplastin reagent including a chromogenic agent (right) versus plasma with saline instead of a chromogenic agent (left).

3B shows a device for detection of bleeding in a surgery procedure.

3C shows a device for detection of bleeding in a surgery procedure.

FIG. 4A-4E present photographic images showing observed fluorescence of substrates after cleavage with thrombin.

4A Shows tube images approx. 3 min after adding substrates with ambient overhead lighting only. Sigma substrate (Sigma-Aldrich, Thrombin Substrate III, Fluorogenic—Calbiochem, Catalog #: 605211

) is shown on the left and Haematologic Technologies (HTI) substrate (Fluorogenic substrate for thrombin (ANSN fluorogenic substrate), Catalog #: SN-20) are shown on the right. Thrombin activity levels are 100, 10 and 0 IU/mL. The solutions were clear with no color change observed in any of the tubes.

4B shows tube images approx. 3 min after adding substrates with ambient overhead lighting and 365 nm flashlight. Sigma substrate is on the left and HTI substrate on the right. Thrombin activity levels are 100, 10 and 0 IU/mL. The only solution displaying fluorescence was the Sigma substrate with 100 IU/mL thrombin.

4C shows tube images approx. 60 min after adding substrates with 365 nm flashlight only (ambient overhead lighting was turned off). Sigma substrate is on the left and HTI substrate on the right. Thrombin activity levels are 100, 10 and 0 IU/mL. The only solution displaying fluorescence was the Sigma substrate with 100 IU/mL thrombin.

4D shows tube images approx. 5 min after combining 100 IU/mL thrombin and substrate. Images were taken under ambient overhead lighting and 365 nm flashlight. Tube on the left is Sigma substrate, tube in the middle is HTI substrate diluted in TBS, and tube on the right is the HTI substrate concentrated in DMSO.

4E shows tube images approx. 5 min after combining 100 IU/mL thrombin and substrate. The image was taken using only the 365 nm flashlight (without any ambient overhead lighting). The Sigma substrate had the strongest fluorescence signal under these lighting conditions (on left, relative rank +++). The fluorescence signal was lowest with HTI substrate diluted in TBS (in the middle, relative rank +). The signal was greater for the other HTI substrate sample (HTI substrate concentrated in DMSO, relative rank ++).

FIG. 5A-5D present photographic images showing observed fluorescence of substrates after cleavage with thrombin.

5A presents a photographic image showing tube images 5 min after combining PNP, substrate and PT reagent (tissue factor and calcium) with ambient overhead lighting only. Tissue factor is the primary activator of physiological clotting response. Tissue factor is present in the PT reagent/thromboplastin. HTI substrate is on the left, Sigma substrate in the middle and Control (no substrate) on the right. The liquid showed the color of plasma and no color change was observed in any of the tubes under ambient lighting.

5B presents a photographic image showing tube images 5 min after combining PNP, substrate and PT reagent (tissue factor and Calcium) with ambient overhead lighting only. The tubes are held at an angle demonstrating that the plasma in the tubes has clotting (thus thrombin has been generated). The liquid is the color of plasma and no color change was observed in any of the tubes under ambient lighting.

5C shows tube images approx. 5 min after combining PNP, substrate and PT reagent (tissue factor and Calcium) with ambient overhead lighting and 365 nm flashlight. HTI substrate is on the left, Sigma substrate in the middle and Control (no substrate) on the right. The only plasma displaying strong fluorescence was the Sigma substrate under these lighting conditions.

5D shows tube images approx. 5 min after combining PNP, substrate and PT reagent (tissue factor and Calcium) with 365 nm flashlight only (no ambient overhead lighting). HTI substrate is on the left, Sigma substrate in the middle and Control (no substrate) on the right. The Sigma substrate in plasma displayed a strong fluorescence signal, while a minor fluorescence signal was observed in the HTI plasma tube under these lighting conditions.

FIG. 6A-6C presents a photographic image showing observed fluorescence in vivo. 6A shows an image of a liver before creating abrasions. 6B shows diffuse/oozing bleeding abrasion defect created in a liver.

6C shows small speckles of fluorescence on oozing defect. Blood can be seen surrounding the fluorescent “spots”.

### DESCRIPTION OF EMBODIMENTS OF THE INVENTION

The invention relates, inter alia, to a method for detecting and localizing a bleeding site in a surgical field e.g. in MIS. It has been realized that the presence of thrombin activity in vivo may indicate bleeding. The invention uses chromogenic or fluorogenic substrate to detect and/or measure thrombin activity in vivo.

Undetected bleeding after surgery procedures are of concern to the medical community. Examples of potential bleeding sites include areas where blood vessels have been ruptured for access e.g. by surgical wound, or by trauma. Examples of potential bleeding sites include areas where a surgeon is actively carrying out a surgical procedure or adjacent thereto. Examples of potential bleeding sites include tissue where a surgeon is actively carrying out a surgical procedure or adjacent thereto. Potential bleeding site include areas around or underneath a clot. Said clot may “open” post-operatively.

An object of the present invention is, inter alia, to provide a method for localizing a site of bleeding in a subject during a surgical procedure e.g. in MIS. The method may be useful e.g. in surgical procedures when visibility is difficult and operator cannot find the bleeding site in order to stop the same. Another object of the present invention is to provide a device for localizing a site of bleeding in a subject during a surgical procedure.

As used herein, the term “localizing” refers, but is not limited to, determining if bleeding is present, detecting bleeding and/or determining the location of bleeding e.g. to pin-point the place of bleeding or the precise place of bleeding. The localization of bleeding is based on the presence of thrombin activity. The term localizing including both possibilities of 1-determining the location or 2-determining if bleeding is present.

Another object of the present invention is to provide a method for determining intensity/severity of bleeding, in a subject during a surgical procedure.

Another object of the present invention is to provide a device for determining intensity/severity of bleeding, in a subject during a surgical procedure.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The method or device in the described exemplary embodiments exploits the presence of thrombin in a bleeding site.

Thrombin is generated at bleeding sites e.g. as a result of blood vessel disruption, and is the final enzyme which activity is required for fibrin clot formation. Thrombin is generated at active bleeding sites through the extrinsic pathway and to a lesser extent the intrinsic pathway.

It was surprisingly found that the presence of thrombin activity could be visualized and consequently bleeding could be detected in vivo using a chromogenic or fluorogenic thrombin substrate. It was surprisingly found that the thrombin activity could be quantified in vivo and consequently bleeding could be quantified using a chromogenic or fluorogenic thrombin substrate. Like fibrinogen and other substrates for thrombin, these chromogenic and fluorogenic substrates can be enzymatically cleaved by the thrombin. Cleavage of a portion of the substrate releases a chromophore or fluorophore which could be visualized, or quantitatively measured using a color or fluorescence analyzer.

The methods and devices disclosed herein are based, inter alia, on the following in vitro and in vivo findings.

In vitro, a change in coloration was evident when thrombin was sprayed on a cellulose matrix coated with a chromogenic substrate of thrombin (S-2238) while change in coloration was not observed when thrombin was sprayed on a non-coated side of the matrix. These findings indicated that thrombin activity could be identified on an absorbent surface coated with a chromogenic substrate.

Also, when a solution containing a chromogenic substrate of thrombin was mixed with different concentrations of thrombin, it was found that the intensity of the color proportionally increased with the amount of thrombin.

A change in color was detected when non diluted plasma activated by a thromboplastin reagent (including calcium) was mixed with a chromogenic substrate of thrombin. The plasma was not diluted prior to the addition of the thromboplastin reagent. The plasma is not considered diluted like it is in other coagulation assays (e.g. factor assays). The results demonstrated that the inherent color of the plasma (straw colored liquid) did not interfere with the detection of thrombin using the chromogenic substrate. The intensity of the color change reflected the activity of thrombin, and accordingly, the amount or concentration of thrombin.

It was found that thrombin fluorogenic substrates were capable of detecting thrombin activity upon exposure to a light source (e.g.) with nominal emission of 365 nm UV light.

Surprisingly, the fluorogenic substrates were capable of detecting thrombin generated in non-diluted pooled normal plasma. For example, a sufficient amount thrombin was generated in pooled plasma by activation of the extrinsic pathway to produce a signal (versus testing with high levels of exogenous thrombin). Particularly, a strong fluorescence signal could be observed with no ambient light and exposure to a 365 nm flashlight.

Improved fluorescence could be detected in a glass tube when nothing blocked or obscured the signal. When attempting to detect fluorescence on a cellulose matrix coated with thrombin, no fluorescence was observed. The fluorescence signal may have been obscured due to the white matrix and non-possible contrast. Alternatively, the cellulose matrix may have quenched the fluorophore preventing visualization. For a better detection of fluorescence on a matrix surface, a non-white matrix may be used for better contrast and/or a matrix made of a material which prevents quenching of the fluorophore (which could impact visualization) may be used. However, surprising results were obtained in vivo. Fluorescence was detected in a canine model with liver and spleen abrasions after spraying the fluorescent substrate on a potential bleeding site.

These results paved the way e.g. to a method of localizing a bleeding site in vivo e.g. during a surgical procedure in a subject comprising: i) introducing a chromogenic or fluorogenic substrate of thrombin into or onto a potential bleeding site in the body of the subject, and ii) detecting a color or fluorescence signal, thereby localizing the bleeding site in said subject.

These results paved the way e.g. to a method of determining the intensity of bleeding during a surgical procedure in a subject, comprising:
i) introducing a chromogenic or fluorogenic substrate of thrombin into or onto a potential bleeding site in the body of the subject, and
   ii) determining the presence and intensity of the color or fluorescence signal, thereby determining the presence and intensity of bleeding.

These results paved the way e.g. to a device for localizing a bleeding site during a surgical procedure in a subject. These results also paved the way e.g. to a device for determining the intensity of bleeding during a surgical procedure in a subject.

The term “potential bleeding site” includes areas where blood vessels (arteries, veins or capillaries) have been punctured for access, or any trauma or surgical wound. Examples of potential bleeding sites include areas where a surgeon is actively carrying out a surgical procedure or adjacent thereto. Examples of potential bleeding sites include tissue where a surgeon is actively carrying out a surgical procedure or adjacent thereto. Examples of potential bleeding site include areas where blood vessels have been ruptured for access e.g. by surgical wound, or by trauma. Potential bleeding site include areas around or underneath a clot. Said clot may “open” post-operatively.

The term “surgical procedure” refers to an action carried out during at least a portion of a medical procedure, such as a medical operation and may refer to other types of medical procedures such as diagnostic procedures and therapeutic procedures.

Surgical procedures where visibility is difficult may be selected from the group consisting of Minimally Invasive Surgery (“MIS”), e.g., endoscopic surgeries such as colonoscopy, laparoscopy, brain endoscopy as well as robot assisted surgery.

As used herein, MIS refers, but is not limited to, a surgery minimizing surgical incisions to reduce trauma to the body. This type of surgery, e.g. laparoscopy, is usually performed using thin-needles and an endoscope to visually guide the surgery.

MIS can include many surgical specialties. Further non-limiting examples of MIS are selected from MIS performed in tumor resections in a cancer surgery, endovascular surgery for treating or repairing an aneurysm, cholecystectomy in a gallbladder surgery, nephrectomy/splenectomy/hepatectomy procedures, and thoracic surgery using video-assisted thoracoscopic surgery (VATS).

The disclosed method may be applied during an intraoperative period. The term “intraoperative” relates to a period that begins when the patient is transferred to the operating room table and ends with the transfer of a patient to the Post Anesthesia Care Unit (PACU). During this period the patient is monitored, anesthetized, prepped, and draped, and the operation is performed. Nursing activities during this period focus on safety, infection prevention, opening additional sterile supplies to the field if needed and documenting applicable segments of the intraoperative report in the patients Electronic Health Record. Intraoperative radiation therapy and Intraoperative blood salvage may also be performed during this time.

The term “substrate” relates to a molecule upon which an enzyme acts. Enzymes catalyze chemical reactions involving the substrate(s). Typically, in the case of thrombin, the substrate binds the thrombin active site, and a thrombin-substrate complex is formed. The substrate is transformed into one or more products, which are then released from the active site. The active site is then free to accept another substrate molecule. A substrate is called ‘chromogenic’ if it gives rise to a colored product when acted on by an enzyme. “Chromogenic” substrate herein also encompasses a luminescent substrate. Similarly, a substrate is called ‘fluorogenic’ if it gives rise to a fluorescent product when acted on by an enzyme.

A chromogenic substrate may bind to the active site of the thrombin enzyme. Once bound, thrombin may cleave (cuts a bond) within the chromogenic substrate releasing a chromophore. A chromophore is a chemical group that absorbs light at a specific frequency and so imparts color to a molecule.

Non limiting examples of chromophores are azo chromophores, anthraquinone chromophores, indigoid chromophores, cationic dyes, polymethine and related chromophores, di- and tri arylcarbenium and related chromophores, phthalocyanine, sulfur compounds, and metal complexes.

A chromophore may be e.g. p-nitroaniline or pNA. The cleavage of the bond causes a difference in absorbance (optical density) between the pNA formed and the original substrate. This optical density change can be monitored visually and appears as a deep yellow coloration. Furthermore, the rate of pNA formation is proportional to the enzymatic activity and enables precise determination of enzyme activity.

Non limiting examples of color include yellow, blue, and green.

In one embodiment, the chromogenic substrate changes to a yellow color once it reacts with thrombin, however red blood cells might obscure or interfere with the visualization of the yellow coloration. Hence, a “filtration technology” may be employed in some embodiments to separate blood cells from plasma, allowing the detection of thrombin without the red blood cell obscuration or interference. Filtration technology typically comprises a thin membrane which separates cells from the plasma. In some embodiments, the matrix is a membrane capable of filtering blood cells e.g. red blood cells (that might interfere with the signal) allowing only the plasma to pass through. The plasma saturating through the membrane may encounter a spot in the membrane where a chromogenic substrate was placed. If thrombin is present in the plasma it may react with the chromogenic substrate in the spot, release the chromophore and produce a stained spot.

A chromogenic substrate that may be used to monitor thrombin activity is e.g. S-2238 (H-D-Phenylalanyl-L-pipecolyl-L-arginine-p-nitroaniline dihydrochloride) which is available from Chromgenix, Instrumentation Laboratory Company, Bedford, Mass., USA. Chromogenic substrates are available for a wide variety of enzymes and are used for determining the activity of those enzymes in in vitro assays. Specifically, for the substrate S-2238, this chromogenic substrate has been used to measure thrombin activity levels in plasma and indirectly measure inhibitory properties of antithrombin III and heparin in benchtop assays. No mention has been made of the use of this chromogenic substrate to detect or measure thrombin activity in vivo.

The concentration of the chromogenic substrate (e.g. S-2238 Chromogenix) may be in the range of about 0.004 mM to about 16.0 mM.

The concentration of the chromogenic substrate (e.g. S-2238, Chromogenix) may be in the range of about 0.04 mM to about 8.0 mM. In one embodiment the chromogenic substrate is luminescent e.g. bioluminescent substrate. An example of a bioluminescent substrate is described by Chen et al., Biosensors and Bioelectronics 77 (2016) 83-89.

A fluorogenic substrate may bind to the active site of the thrombin enzyme. Once bound, thrombin may cleave (e.g., breaks a bond) within the fluorogenic substrate releasing a fluorophore.

Different fluorescent molecules can be attached to the thrombin substrate. E.g. fluorescent molecules having an emission energy modulation over a broad domain of the visible and near-infrared (NIR) spectrum 650-1000 nm or 600-850 nm. For example NIR light at 800 nm upon optical excitation at 780 nm (e.g. Ghoroghchian et al. “In vivo fluorescence imaging: a personal perspective” Wiley Interdiscip Rev Nanomed Nanobiotechnol. 2009; 1(2): 156-167).

Thrombin activity may be determined by using a fluorogenic substrate that e.g. binds to the active site of the thrombin enzyme.

Fluorescence is the emission of light by a substance that has absorbed light or other electromagnetic radiation. In most cases, the emitted light has a longer wavelength, and therefore lower energy, than the absorbed radiation. The most striking example of fluorescence occurs when the absorbed radiation is in the ultraviolet region of the spectrum, and thus invisible to the human eye, while the emitted light is in the visible region, which gives the fluorescent substance a distinct color that can be seen when exposed to UV light. A relatively faint fluorescence signal (having a low number of emitted photons) can be observed e.g. against a low-noise background.

A fluorescence spectrophotometer (also referred to as a fluorometer) may be used to measure the fluorescence signal.

Typically, a fluorometer looks like a standard spectrophotometer, and uses square cuvettes in which the light does not pass through the sample onto an inline detector. The detector is at a 90-degree angle. The fluorometer has a light source and a filter or monochromator to select a defined excitation wavelength, which is then directed into a sample. The light emitted from the sample is then passed through another filter or monochromator which selects the emission wavelength of interest as well as removing most of the excitation light before being measured by a detector.

The detection system may also be carried out by using a e.g. a UV flashlight that emits light at a specific wavelength and observing for the fluorescence signal or any other detection system. For each substrate, the optimal excitation light wavelength may be used.

The thrombin activity in a sample may be expressed in units/ml, calculated by comparison of the relative fluorescent rate of the sample with that of the reference thrombin, using fluorogenic substrates e.g. using two substrates e.g. Sigma and HTI substrates described herein.

Estimating the amount of thrombin at a bleeding site is extremely complex. For example, the amount of thrombin at a bleeding site may depend on the amount of tissue factor (TF) exposed, blood flow, available platelets, amounts of procoagulant clotting factors, and amounts of endogenous anticoagulants. Tissue factor (TF) is the primary activator of physiological clotting response.

Oftentimes, as little as 1 IU of thrombin can cause a fibrin clot to form. When there is sufficient tissue damage (and exposure of TF), thrombin generation and clotting can occur quickly under optimal conditions (e.g. in 10-15 sec based on PT clotting times), in other circumstances, clotting can take much longer.

In general, thrombin activity is a function of bleeding level.

The intensity of bleeding may be determined on a relative basis. For example, the thrombin substrate may be in two concentrations, i.e. low and high. If a signal is detected only using the high concentration of substrate, this would represent a relatively lower thrombin activity compared with the signal detected using both the high and low substrate concentrations.

Advantageously, the substrate for use in vivo is biocompatible.

Non limiting example of fluorogenic substrates is: Thrombin Substrate III (Sigma-Aldrich), and Fluorogenic (Calbiochem, Catalog #: 605211. Excitation max.: 360-380 nm; emission max.: 440-460 nm).

Additional non-limiting example of fluorogenic substrates is: Fluorogenic substrate for thrombin (ANSN fluorogenic substrate Catalog #: SN-20. Excitation=352 nm, Emission=470 nm, Haematologic Technologies, Inc.)

Different fluorescent molecules can be attached to the thrombin substrate, such as molecules disclosed in Ghoroghchian et al. “In vivo fluorescence imaging: a personal perspective” Wiley Interdiscip Rev Nanomed Nanobiotechnol. 2009; 1(2): 156-167).

The concentration of the fluorogenic substrate may be in the range of about 0.0004 mM to about 10.0 mM .

The concentration of the fluorogenic substrate may be in the range of about 0.004 mM to about 7.0 mM.

The substrate may be dissolved in water or blood allowing its reaction with thrombin on the tissue.

In some embodiments, substrate is first solubilized in dimethyl sulfoxide (DMSO), since DMSO is miscible in water or blood. The substrate in DMSO (sometimes diluted with aqueous solution such as a buffer solution) is delivered onto the tissue and reacts with thrombin, if present on the tissue.

Although DMSO is commonly used as a dispersant to solubilize fluorescent substrates, other solvents may be used, including other relatively harmless solvents like ethanol.

The sensitivity of the reaction and signal can be optimized when using a fluorescent substrate. Increased sensitivity with a fluorescent substrate may require specialized equipment. The signal specificity may be increased by using an optimal light source and/or glasses that enhance visualization of the emission wavelength by, for example, suppressing transmission of other visible wavelengths. Other image enhancement methods could be used such as real-time image processing in conjunction with photographic or video imaging.

Typically, the membrane in the “filtration technology” comprises at least one of a chemical or natural fiber. The fiber may be selected from one or more of glass fiber, polyester, nitrocellulose, polysulfone, and cellulose. The membrane allows to separate the plasma which could potentially contain thrombin from the rest of the whole blood components. Upon such a separation, the interference from the blood cells, particularly the red blood cells, with the signal is minimalized. A commercially available example of such a membrane is Vivid Plasma Separation Membrane (Pall Life Sciences, Port Washington, N.Y., USA).

Fibrin clot formation on a matrix, membrane or surface during the detection step may limit the ability of the thrombin to absorb or come into contact with the substrate. To prevent such clotting, an inhibitor of fibrin polymerization can be used. For example, the tetra-peptide Gly-Pro-Arg-Pro (GPRP) may be used. GPRP can prevent clot formation by blocking fibrin monomer polymerization and keep the blood/plasma in a liquid state thus allowing the thrombin to interact with the chromogenic substrate resulting in a color change.

In non-limiting examples, one or more fibrin polymerization inhibitors, may be added on a matrix containing a chromogenic or fluorogenic substrate. Also, one or more fibrin polymerization inhibitors may be added to a solution comprising a solubilized chromogenic or fluorogenic substrate.

A list of fibrin polymerization inhibitors can be found in U.S. Pat. No. 10,357,589.

It can be challenging for a surgeon to localize bleeding, e.g. oozing bleeding, especially during laparoscopic/endoscopic procedures. While viewing a procedure on a video screen during an MIS procedure, the ability for the surgeon to localize bleeding, e.g. oozing, in the surgical field is not trivial. Capability to localize bleeding is critical e.g. in the decision if and where to use a hemostatic product and what kind of hemostatic product.

Typically, topical hemostatic agents are used as an adjunct method to control bleeding when standard methods are ineffective or impractical.

Advantageously, the method and/or device disclosed herein allows a qualitative assessment of whether there is bleeding or not.

Advantageously, the method and/or device disclosed herein allows to distinguish an active bleeding from clotted blood.

Advantageously, the method and/or device disclosed herein allows quantifying the amount and/or intensity of a bleeding in vivo.

If bleeding is present and detected at a site of tissue injury, adjunctive hemostatic agents could be used to help to stop or minimize the loss of blood. Alternatively, if no bleeding is present, the use of hemostatic device and biologics could be minimized.

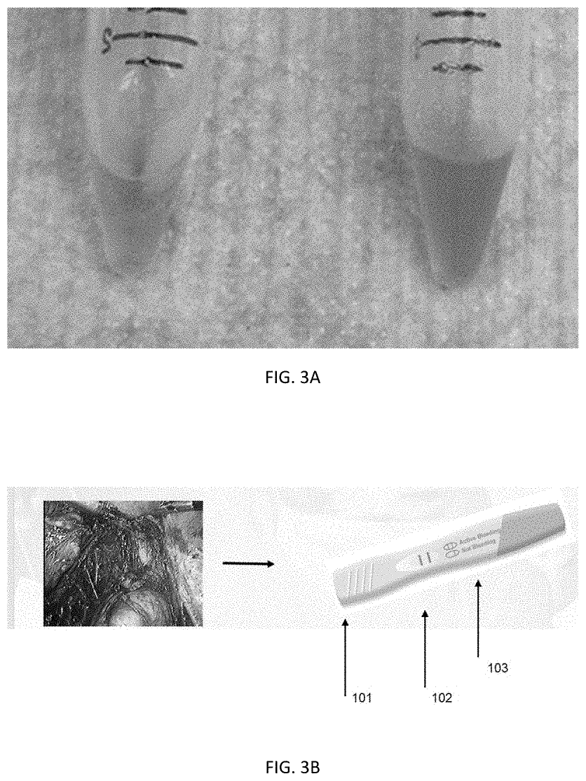

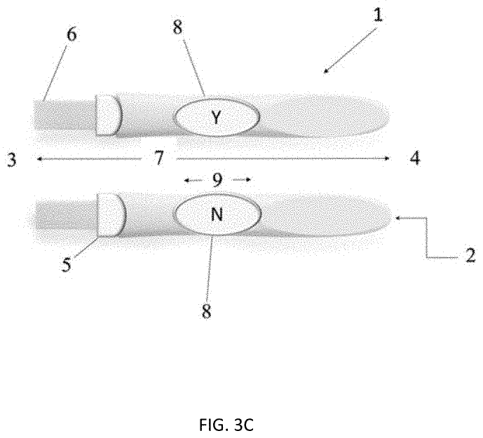

Provided is an exemplary configuration of test device (1) as in FIG. 3C comprising:

A housing (2) having distal end (3) and proximal end (4); the distal end (3) may have an opening (5); device (1) may have a matrix (6). Matrix (6) may be contained within an area (7) defined between proximal end (4) and distal end (3) of said housing (2). At least part of matrix (6) may comprise a chromogenic or fluorogenic thrombin substrate (test area). The matrix (6) may be capable of adsorbing liquid from blood up to the substrate, allowing the thrombin from blood (if present) to react with the substrate to produce a visual fluorogenic or chromogenic signal (8). Housing (2) may have a detection area (9) disposed in the housing to visualize the signal.

In one embodiment , housing (2) is made of plastic.

Opening (5) may be present where distal end (3, FIG. 3B 101) of the matrix (e.g. membrane) is located. The distal end of the matrix may protrude from the opening. The protruding distal end of the matrix may be used to touch a potential bleeding site.

The matrix is configured to wick the blood plasma to the substrate's location. If thrombin is present in the plasma it will react with the substrate and create a colored or fluorescent product.

The signal may be visually detected (with light or UV source,).

Test device may have legend/key (FIG. 3B 103) allowing to determine whether thrombin activity is present based on the results in detection area (9, FIG. 3B 102).

Presence of thrombin activity is indicative of bleeding.

In general, thrombin activity is a function of bleeding level.

In one embodiment, the device is of a size of about 15 cm long, and 1-2 cm wide and 1 cm deep.

At least part of the matrix may also comprise dry thrombin allowing to provide a positive control. Alternatively, housing (2) may include another matrix (a control matrix) including dry thrombin serving as a positive control.

The matrix may be capable of drawing liquid from blood from the opening in distal end (3, FIG. 3B 101) through which liquid from blood is capable of being drawn e.g. by capillarity action to a chromogenic substrate of thrombin which is present in the matrix at the testing area.

As provided above, housing (2) may include a detection area (9, FIG. 3B 102). Detection area (9, FIG. 3B 102) may be positioned in or near the proximal end (4) of housing (2) allowing, after reaction of thrombin from blood with the substrate, visually detecting e.g. at or near proximal end (4) of the housing at least one signal.

The signal may be “printed” as a pattern or word to indicate a positive result.

The device may contain at the distal end of the housing a hydrophilic porous septum, membrane which covers an opening in the distal end through which liquid from blood is capable of being drawn.

In one embodiment, the hydrophilic porous septum, membrane is impermeable to one or more blood cells such as red blood cells.

In one embodiment, the device comprises a casing for housing components of the device, the casing being capable of shielding from external light sources.

In operation, the casing is removed.

Distal end (where the matrix is located) may be placed to touch onto a potential bleeding site, blood or fluid in the surgical field. The matrix may wick the blood plasma to the substrate's location and the produced signal (8), if thrombin is present, a signal (8) may be visually detected visually in the detecting area (9) e.g. with light or UV source (e.g., flashlight). Detection of a signal, is indicative of presence of thrombin activity and detection of bleeding.

The matrix may be hydrophilic, absorbent, porous, biocompatible, and/or non-adhesive. Typically, the matrix is one that does not potentiate or induce clotting (e.g. due to intrinsic pathway activation).

Non limiting example of matrices may be hydrophilic wound dressing materials or felts; cellulosic (e.g. gauze, and cottonoids); polyurethane sponge (e.g. Hydrasorb); PG910 could be a viable candidate.

The disclosed methods and devices provide one or more of the following advantages: allows to determine whether there is a leak of blood from the target site (e.g. tissue) identification of bleeding versus already clotted blood within the surgical field, minimize time for visualization of bleeding thus decreasing procedural times, provides adequate visualization reducing mechanical trauma, prevents greater bleeding due to mis-identification of structures, and prevents misidentified anatomical structures, thus providing an improved way to visualize and protect anatomical structures in the surgical field. The detection of bleeding would be helpful, inter alia, in making the decision to treat a potential bleeding site with a hemostatic agent.

A target site may be at the area where the surgeon is actively carrying out the surgical procedure tissue or adjacent thereto.

The disclosed methods and device provide the above advantages inter alia in a MIS and/or open surgical field.

The in vivo method or device in the described exemplary embodiments may be used for constant monitoring of potential bleeding sites for critical re-bleeding, and for alerting medical staff if bleeding is detected. The in vivo method and device is capable of providing constant monitoring during surgery.

The terms “comprises”, “comprising”, “includes”, “including”, “contains”, “containing”, “has”, “having”, and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an”, and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, analytical, pharmacological, biological, biochemical and medical arts.

As used herein, and unless stated otherwise, the terms “by weight”, “w/w”, “weight percent”, or “wt. %”, which are used herein interchangeably describe the concentration of a particular substance out of the total weight of the corresponding mixture, solution, formulation or composition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Detection of Thrombin Activity on Chromogenic Substrate Coated onto Surface

S-2238 chromogenic thrombin substrate ((H-D-Phenylalanyl-L-pipecolyl-L-arginine-p-nitroaniline dihydrochloride) which is available from Chromgenix, Instrumentation Laboratory Company, Bedford, Mass., USA catalog number: 82032439), 25 mg, was dissolved in 12.5 mL of water. The resulting concentration was 2 mg/mL (25 mg in 12.5 mL). 1 mL of 2 mg/mL of the chromogenic substrate was sprayed onto one side of a white cellulose matrix (paper towel Scott C-Fold) approx. 4 in×6 cm in using the EVICEL® ASA tip (product code 3921S). The other side was not coated or sprayed with the substrate.

A kit of EVICEL® (product code 3905) was thawed in a 37° C. water bath for 5 min. To evaluate whether a small amount of thrombin could be detected, a drop (50-100 uL) of thrombin was placed on a fingertip and spread onto the cellulose matrix. This was repeated on the substrate coated side and also applied in duplicate on the non-substrate coated side. After 30 sec at room temperature, a yellow coloration could be seen on the cellulose matrix on the substrate coated side, but not on the un-coated side.

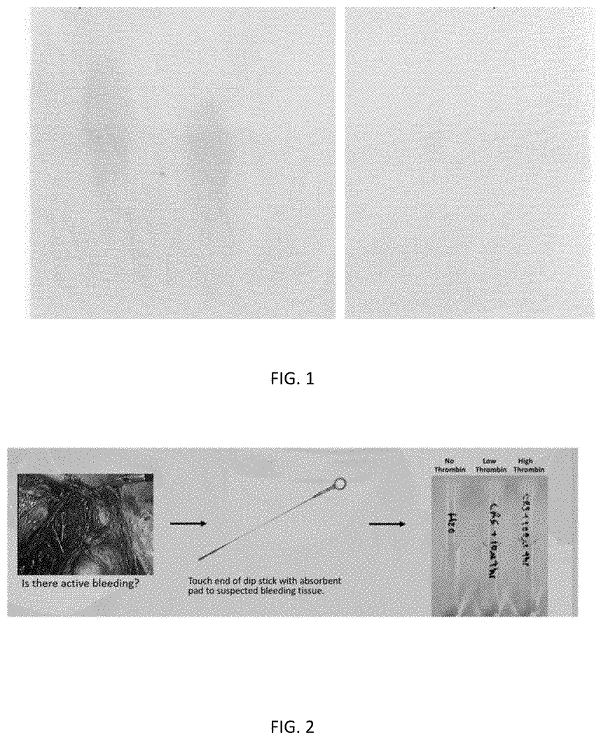

A yellow coloration (dark streaks) was evident on the substrate coated side of the matrix (FIG. 1) while no yellow was observed on the non-coated side. This indicates that thrombin activity could be identified using a substrate-coated absorbent surface (i.e. cellulose matrix). As a control, thrombin was applied to the non-coated side of the matrix and no color changed was observed.

### Example 2

### Thrombin Activity on Chromogenic Substrate using a Dip Stick

A “dip stick” can be dabbed onto a tissue surface that is suspected of bleeding (FIG. 2). The end of the dip stick can then be placed into a solution containing the visualization agent (chromogenic substrate). The amount of the thrombin present in the dip stick is determined by comparison with a standard curve obtained by running similar tests with known thrombin concentrations.

For example, 0.2 mL of the 2 mg/mL chromogenic substrate was combined with 1.8 mL of water in a 12×75 cm in 2 borosilicate glass tubes. The solution was gently mixed to distribute the substrate. A third tube was filled with 2 mL of water as an untreated control. To one of the tubes with the diluted substrate, 100 uL of 1000 IU/mL thrombin was added and 10 uL was added to the other tube. After about 2 min at room temperature, a photo was captured of the tubes.

The tube with the 100 uL of 1000 IU/mL thrombin showed a yellow coloration, while the tube with 10 uL thrombin showed a faint yellow coloration. The tube with water is shown as a reference. The intensity of the color change reflected the amount of thrombin.

According to the intensity of the color in the solution containing the visualization agent (chromogenic substrate) in which the “dip stick” was placed it is possible to detect bleeding and the amount of thrombin and therefore also the intensity of bleeding in the suspected bleeding site.

### Example 3

### Detection of Chromogenic Substrate Product in Plasma

Plasma has a background color (straw color). To test whether the product of the chromogenic substrate can be detected in plasma, 0.4 mL pooled normal plasma (PNP from George King Biomedical) was activated by a thromboplastin reagent (0.2 mL, Neoplastin CL plus product number 00375) with calcium to allow thrombin generation.

Either 0.2 mL saline (as a control) or 0.2 mL of 2 mg/mL of the chromogenic substrate was added to the tube and mixed on the tube rocker.

Evidence of clot formation was present after approx. 30 sec at room temperature. In view of the presence of fibrinogen in plasma, complete clot formation, i.e. solid/stable clot, occurred at approx. 2 min at room temperature. The yellow coloration/hue was significantly stronger for the clotted plasma with chromogenic substrate compared with the saline control (FIG. 3A).

This study demonstrates that the inherent color of the plasma (straw colored liquid) does not interfere with the detection of thrombin using the chromogenic substrate. The intensity of the color change reflects the amount of thrombin.

In one embodiment the device comprises a casing for housing components of the device, the casing being capable of shielding from external light sources.

Provided is a test device as in FIG. 3B. 101 represents the area where blood or fluid is absorbed onto the substrate, 102 represents the detection area (reading area) where thrombin in the absorbed blood or fluid react with the chromogenic/fluorogenic substrate, 103 represents the legend/key to determine whether thrombin activity or thrombin (bleeding) is present based on the results in the detection area 102.

In exemplary configuration, these test devices are sized to fit the practitioners' hand size (about 15 cm long, 1-2 cm wide and 1 cm deep) and are made of plastic.

### Example 4

### Detection of Thrombin in Solution with Fluorescent Substrates

Previous studies focused on chromogenic substrates to detect thrombin activity, in this in vitro study the ability of fluorogenic substrates to detect thrombin activity was assessed. The two fluorogenic substrates that were evaluated were purchased from Haematologic Technologies and Sigma-Aldrich. Testing included detection of thrombin in solution, on a surface (matrix) and in plasma.

Sigma-Aldrich Substrate: Sigma-Aldrich, Thrombin Substrate III, Fluorogenic—Calbiochem, Catalog #: 605211. Packaging: ampoule/bottle with 25 mg lyophilized powder. Excitation max.: 360-380 nm; emission max.: 440-460 nm; Molecular weight 718 D; Solubility in DMSO (5 mg/ml) from product information.

5 mg substrate was placed in light impermeable dark amber 2.0 mL microfuge tube. 1.0 mL of DMSO was added to the substrate to create 5 mg/mL stock solution in the microfuge tube and was mixed by gentle inversion.

HTI Substrate: Haematologic Technologies, Inc., Fluorogenic substrate for thrombin (ANSN fluorogenic substrate), Catalog#: SN-20. Packaging: 10 mM in vial/microcentrifuge tube; 1 mg in each vial at 7.5 mg/mL, thus 0.133 mL/vial in DMSO. Excitation=352 nm, Emission=470nm. Substrate provided as 10 mM stock solutions in DMSO, generally used in range of 400 nM for in vitro diagnostics.

0.050 mL from manufacture's vial were removed and dispensed into light impermeable dark amber 2.0 mL microfuge tubes containing 1.5 mL tris buffered saline (TBS), pH 7.4, to create stock substrate solution. Tris concentration was 20 mM and sodium chloride concentration was 150 mM. This represents a 1:30 dilution of the manufactures stock (0.375 mg in 1.5 mL is 0.25 mg/mL).

Thrombin: EVICEL® thrombin from fibrin sealant.

DMSO: Sigma-Aldrich, Dimethyl Sulfoxide, product number 296147-25G.

Light Source: JowBeam flashlight, which has a nominal emission of 365 nm wavelength of light, was used for fluorescence visualization.

Testing Procedure and Results:

Detection of Thrombin in Solution

1. 2 mL of 0, 10 IU/mL and 100 IU/mL thrombin in saline (none, low, and high thrombin) was prepared in duplicates and tubes were labeled accordingly. Stock of EVICEL® thrombin is about 1000 IU/mL.

2. A defined amount of thrombin substrate was added to thrombin solutions as follows and fluorescence of the mixture was evaluated. The substrate stock solutions were diluted 1:10 in Tris Buffered saline (TBS) buffer (180 uL TBS and 20 uL of stock substrate solution). TBS buffer comprises 20 mM Tris and 150 mM sodium chloride, and is adjusted to pH 7.4. 0.05 mL of the diluted stock solution (1:10 dilution) was added to the 2 mL of thrombin prepared in step 1, mixed at room temperature, and incubated for about 3 mins.

3. Images of tubes were captured while shining with light (using various light emitting sources) and responses were compared for different amounts of thrombin.

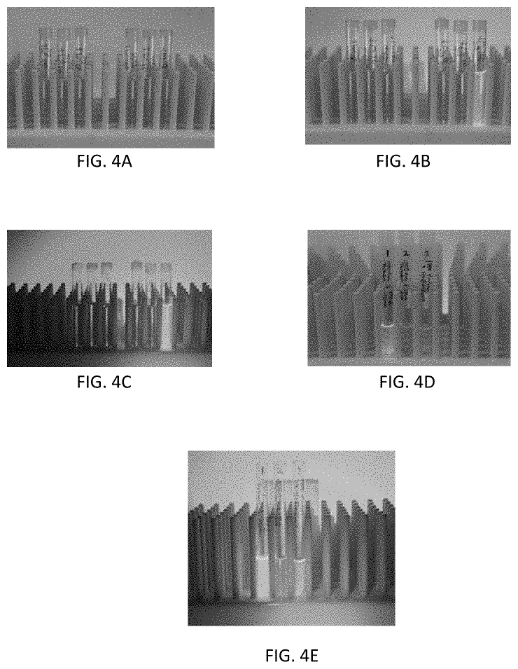

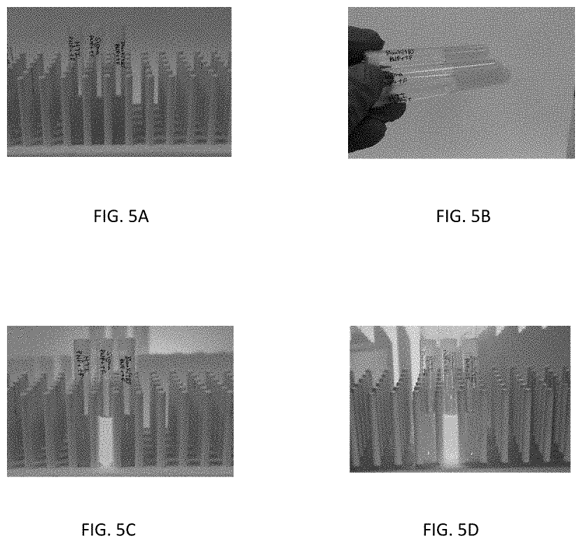

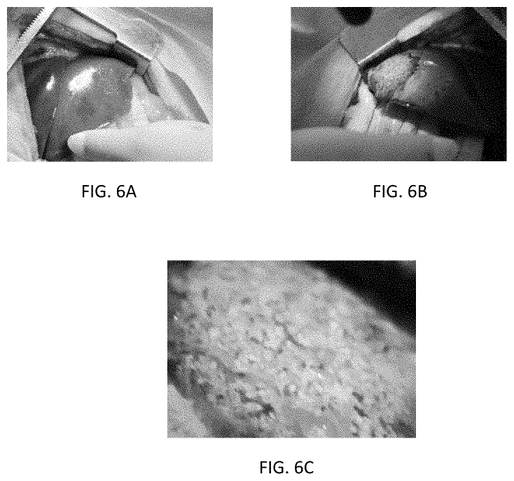

FIG. 4A shows tube images approx. 3 min after adding substrates with ambient overhead lighting only. Sigma substrate is shown on the left and HTI substrate is shown on the right. Thrombin activity levels are 100, 10 and 0 IU/mL. The solutions were clear with no color change observed in any of the tubes.

FIG. 4B shows tube images approx. 3 min after adding substrates with ambient overhead lighting and 365 nm flashlight. Sigma substrate is shown on the left and HTI substrate on the right. Thrombin activity levels are 100, 10 and 0 IU/mL. The only solution displaying fluorescence was the Sigma substrate with 100 IU/mL thrombin. UV blocking glasses were worn throughout the visualization procedures when the 365 nm flashlight was used.

FIG. 4C shows tube images approx. 60 min after adding substrates with 365 nm flashlight only (ambient overhead lighting was turned off). Sigma substrate is shown on the left and HTI substrate on the right. Thrombin activity levels are 100, 10 and 0 IU/mL. The only solution displaying fluorescence was the Sigma substrate with 100 IU/mL thrombin. The fluorescence was more intense appearing with the ambient lights off. There did not appear to be a change in intensity between 3 min and 60 min (suggesting that all of the substrate was cleaved within 3 min).

Since the HTI fluorogenic substrate did not seem to work in the earlier study (perhaps due to the relatively low concentration used), the study was repeated with higher amounts of HTI substrate. 100 IU/mL of thrombin was used.

To 1.0 mL of 100 IU/mL thrombin in saline, 100 uL of Sigma substrate at 5 mg/mL in DMSO was added [500 ug total substrate].

To 1.0 mL of 100 IU/mL thrombin in saline, 100 uL of HTI substrate diluted in TBS at 0.25 mg/mL (50 uL HTI manufacture's stock and 1.5 mL TBS, i.e. 1:30 dilution) was added [25 ug total substrate].

To 1.0 mL of 100 IU/mL thrombin in saline, 10 uL of HTI manufacture's stock (7.5 mg/mL in DMSO) was added [75 ug total substrate]

Images of tubes were captured under various lighting conditions (using various light sources) and responses were compared.

The experiments were carried out at room temperature.

FIG. 4D shows tube images approx. 5 min after combining 100 IU/mL thrombin and substrate. Images were taken under ambient overhead lighting and 365 nm flashlight. Tube on the left is Sigma substrate, tube in the middle is HTI substrate diluted in TBS, and tube on the right is the HTI substrate concentrated in DMSO.

As previously seen, the Sigma substrate had the strongest fluorescence signal under these lighting conditions (on left). The fluorescence signal was lowest with HTI substrate diluted in TBS (in the middle). The signal was greater for the other HTI substrate sample (HTI substrate concentrated in DMSO).

FIG. 4E shows tube images approx. 5 min after combining 100 IU/mL thrombin and substrate.

The image was taken using only the 365 nm flashlight (without any ambient overhead lighting). Tube on the left is Sigma substrate, tube in the middle is HTI substrate diluted in TBS, and tube on the right is the HTI substrate concentrated in DMSO.

The Sigma substrate had the strongest fluorescence signal under these lighting conditions (on left, relative rank +++). The fluorescence signal was lowest with HTI substrate diluted in TBS (in the middle, relative rank +). The signal was greater for the other HTI substrate sample (HTI substrate concentrated in DMSO, relative rank ++).

The results show that apparently the differences in fluorescence intensities are related to the amount of substrate in each tube. The absolute amount of each substrate was: Sigma 500 ug substrate, HTI in TBS 25 ug substrate, and HTI in DMSO 75 ug substrate.

### Example 5

### Detection of Thrombin on a Surface

2 mL solutions of 0, 10 IU/mL and 100 IU/mL thrombin in saline (none, and low and high thrombin) were prepared using a stock solution of EVICEL® thrombin of about 1000 IU/mL. 1 mL of each thrombin dilution was sprayed onto white cellulose matrix (paper towel Scott C-Fold) in a designated area.

Immediately a defined amount of thrombin substrate, prepared as follows, was sprayed on the thrombin and coated cellulose matrix and fluorescence of the mixture was evaluated. The substrate stock solutions were diluted 1:10 in TBS (using 900 uL TBS and 100 uL of stock substrate solution). 1.0 mL of the diluted stock solution (1:10 dilution) was sprayed across all three regions of the cellulose matrix coated with thrombin at different levels.

Images of cellulose matrix under various lighting conditions (with various light sources) were captured and the response was compared for different amounts of thrombin and substrates. The Experiments were carried out at room temperature.

Approx. 3 min after spraying cellulose matrices with substrates with ambient overhead lighting and 365 nm flashlight, no color changes or fluorescence was observed on any of the cellulose matrices. Thrombin activity levels are 100, 10 and 0 IU/mL (of note, in previous experiments carried out in solution, the only solution displaying fluorescence was the Sigma substrate with 100 IU/mL thrombin), the fluorescence may have been obscured because of the white cellulose matrix surface and no contrast was possible.

Approx. 10 min after spraying substrates with 365 nm flashlight only (no ambient overhead lighting), no fluorescence was observed on any of the cellulose matrices. The fluorescence may have been obscured due to the white cellulose matrix surface and no contrast was possible.

Approx. 60 min after spraying Sigma substrate with 365 nm flashlight only (no ambient overhead lighting), after moving the cellulose matrix out of its original position, spots of fluorescence could be seen on the surface underneath the cellulose matrix especially under the 100 IU/mL thrombin with the Sigma substrate. Apparently, the thrombin soaked through the cellulose matrices, perhaps in high concentration and combined with the Sigma substrate. Alternatively, the cellulose matrices may have a quencher that prevents fluorescence visualization.

Approx. 60 min after spraying HTI substrate with 365 nm flashlight only (no ambient overhead lighting). After moving the cellulose matrices out of its original position, no spots of fluorescence could be seen. This is in contrast to the Sigma substrate where evidence of fluorescence could be seen.

### Example 6

### Detection of Thrombin Generated in Plasma

Pooled normal plasma (PNP) was purchased from George King Biomedical was thawed at 37 C for 5 min. 0.5 mL of PNP was aliquoted into 3-10×75 mm borosilicate glass tubes. To each to glass tube, 0.10 mL stock fluorescent substrate or 0.10 mL stock TBS was added. Tubes were labeled accordingly. Sigma substrate stock had a concentration of 5 mg/mL in DMSO. HTI substrate stock had a concentration of 0.25 mg/mL in TBS.

1.0 mL of PT reagent (Diagnostica Stago, STA Neoplastine Cl Plus containing calcium) was dispensed into the three tubes.

Images of tubes were captured while using shining light (using various light sources) and response was compared for different substrates.

Clotting results:

The blank/TBS control clotted in approx. 10 sec (as expected for a PT clotting time).

The HTI substrate sample clotted also in approx. 10 sec.

The Sigma substrate sample remained fluid for approx. 3 min before clotting. Perhaps the higher concentration of DMSO inhibited the clotting. Photos were taken at 5 min after addition of the PT reagent and ambient room light and the 365 nm flashlight was used. See photo captions for additional details.

The Experiments were carried out at room temperature.

FIG. 5A: shows tube images 5 min after combining PNP, substrate and PT reagent (containing tissue factor and calcium) with ambient overhead lighting only. HTI substrate on the left, Sigma substrate in the middle and Control (no substrate) on the right. The liquid showed the color of plasma and no color change was observed in any of the tubes.

FIG. 5B: Tube images 5 min after combining PNP, substrate and PT reagent (TF and Calcium) with ambient overhead lighting only. The tubes are held at an angle demonstrating that the plasma in the tubes has clotting (thus thrombin has been generated). The liquid is the color of plasma and no color change was observed in any of the tubes under ambient lighting.

FIG. 5C: shows tube images approx. 5 min after combining PNP, substrate and PT reagent (TF and Calcium) with ambient overhead lighting and 365 nm flashlight. HTI substrate on the left, Sigma substrate in the middle and Control (no substrate) on the right. The only plasma displaying strong fluorescence was the Sigma substrate under these lighting conditions.

FIG. 5D: shows tube images approx. 5 min after combining PNP, substrate and PT reagent (TF and Calcium) with 365 nm flashlight only (no ambient overhead lighting). HTI substrate on the left, Sigma substrate in the middle and Control (no substrate) on the right. The Sigma substrate in plasma displayed a strong fluorescence signal, while a minor fluorescence signal was observed in the HTI plasma tube under these lighting conditions. The difference in fluorescence signal may be due (at least in part) to the higher concentration of the Sigma substrate (5 mg/mL) added to the plasma, while the HTI substrate was at 0.25 mg/mL.

Since the HTI fluorogenic substrate didn't seem to work in the earlier study (perhaps due to the relatively low concentration used), the study was repeated with higher amounts of HTI substrate. 100 IU/mL of thrombin was used.

To 1.0 mL of 100 IU/mL thrombin in saline, 100 uL of Sigma substrate at 5 mg/mL in DMSO was added [500 ug total substrate].

To 1.0 mL of 100 IU/mL thrombin in saline, 100 uL of HTI substrate diluted in TBS at 0.25 mg/mL (50 uL HTI manufacture's stock and 1.5 mL TBS, i.e., 1:30 dilution) was added [25 ug total substrate].

To 1.0 mL of 100 IU/mL thrombin in saline, 10 uL of HTI manufacture's stock (7.5 mg/mL in DMSO) was added. [75 ug total substrate]

images of tubes were captured while using shining light (using various light sources) and response was compared.

The experiments were carried out at room temperature.

The results show that: The thrombin fluorogenic substrates were capable of detecting thrombin in solution. Importantly, the fluorogenic substrates were capable of detecting thrombin generated from pooled normal plasma, i.e., enough thrombin was generated in the plasma from activation of the extrinsic pathway to produce a signal (versus testing with high levels of exogenous thrombin). The fluorescence intensity observed with the Sigma substrate was greater than that with the HTI substrate likely due to the amount of substrate used in these studies. Typically for in vitro studies, the amount of fluorogenic substrate needed is minimal (1000 or more fold dilution is used for in vitro studies), however, a fluorescence spectrophotometer is used to measure the signal. The best fluorescence signal was observed with the ambient light off using 365 nm flashlight

The ability to detect a fluorescence signal was most optimal when viewing the signal in a glass tube since there is nothing blocking or obscuring the signal.

### Example 7

### In Vivo Testing

In a preceding experiment it was shown that fluorescence could not be seen on a surface. In a preceding experiment it was shown that fluorescence could be seen on a on a glass tube without background color. In vitro studies with fluorogenic substrates demonstrated that thrombin could be detected, including thrombin generated in plasma through the extrinsic pathway.

In this in vivo study, the ability to detect thrombin on a surface in a live animal after creating a bleeding defect was assessed in a canine model with liver and spleen abrasions.

Diffuse/oozing bleeding abrasion defect was created using cautery tip cleaning pad.

0.5 mL of Sigma fluorogenic substrate (Stock solution 5 mg/mL concentration in DMSO protected from light) was sprayed onto bleeding site and visualized with flashlight (356 nm) 2-3 min after application.

Close-up showing small speckles of fluorescence on oozing defect. Blood can be seen surrounding the fluorescent “spots”.

A fluorescence signal could be detected at bleeding site—no exogenous thrombin added.

The sigma substrate enabled detection of bleeding in a liver abrasion model, though not in more challenging models.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method of localizing a bleeding site during a surgical procedure in a subject comprising:
i) introducing a chromogenic or fluorogenic substrate of thrombin into or onto a potential bleeding site in the body of the subject, and
ii) detecting a color or fluorescence signal,
thereby localizing the bleeding site in said subject.

2. A method of determining the intensity of bleeding during a surgical procedure in a subject, comprising
i) introducing a chromogenic or fluorogenic substrate of thrombin into or onto a potential bleeding site in the body of the subject, and
ii) determining the presence and intensity of the color or fluorescence signal, thereby determining the presence and intensity of bleeding.

3. The method according to claim 1 wherein said chromogenic or fluorogenic substrate is:
(i) immobilized on a membrane or on a porous matrix; or
(ii) sprayed directly on the potential bleeding site; or
(iii) introduced intravenously; or
(iv) otherwise systemically introduced.

4. The method according to claim 1 or 2, being devoid of an exogenous step of generating thrombin.

5. The method of claim 2, for detecting an intensity of bleeding selected from the group consisting of oozing/mild bleeding and severe/challenging bleeding.

6. The method according to claim 1 or 2, wherein the surgical procedure is minimal invasive surgery (MIS).

7. The method according to claim 1 or 2, wherein the substrate is fluorogenic.

8. The method according to claim 1 or 2, wherein the substrate is chromogenic.

9. The method according to claim 1 or 2, wherein the substrate is immobilized on a matrix.

10. The method according to claim 1 or 2 , wherein the substrate is immobilized on a matrix and the matrix is a membrane that is impermeable to red blood cells.

11. A device (1) for detection of bleeding in a surgery procedure, comprising:
a housing (2) having distal end (3, 101) and proximal end (4); wherein the distal end (3, 101) is configured to contact blood in a potential bleeding site of a subject;
a matrix (6) contained within an area (7) defined between said proximal (4) and distal (3, 101) end of said housing, wherein at least part of said matrix comprises a chromogenic or fluorogenic thrombin substrate and said matrix is capable of adsorbing a liquid being present in the blood up to the substrate, allowing thrombin if present in the liquid to react with the substrate to produce a visual fluorogenic or chromogenic signal (8); and
a detection area (9) disposed in the housing and configured to visualize the signal.

12. The device according to claim 11, wherein said distal end (3, 101) has an opening (5).
